(19) United States
(12) Patent Application Publication
(10) Pub. No.: US 2026/0256714 A1
(43) Pub. Date: Sep. 3, 2026

(54) PHARMACEUTICAL COMPOSITION FOR PREVENTING OR TREATING METABOLIC DISEASES CONTAINING DEMETHYLZEYLASTERAL

(71) Applicant: SEOUL NATIONAL UNIVERSITY R&DB FOUNDATION, Seoul (KR)

(72) Inventors: HEE YANG, Seoul (KR); SEO HYUN SHIN, Seoul (KR); GI HYUN HUR, Seoul (KR); NA RA KIM, Gyeonggi-do (KR); JOON YOUNG LEE, Seoul (KR); YE EUN KIM, Seoul (KR); JUNG HAN YOON, Seoul (KR); KI WON LEE, Seoul (KR)

(21) Appl. No.: 18/872,978

(22) PCT Filed: Mar. 9, 2023
(86) PCT No.: PCT/KR2023/003227

§ 371 Date: Dec. 9, 2024

(30) Foreign Application Priority Data
Jun. 7, 2022 (KR) 10-2022-0069114

(51) Int. Cl. A61K 31/122; A61K 36/37

(52) U.S. Cl. CPC A61K 31/122; A61K 36/37

(57) ABSTRACT

A composition contains demethylzeylasteral represented by Formula 1 or a pharmaceutically acceptable salt thereof. The composition is a glucocorticoid receptor antagonist that inhibits intracellular glucocorticoid receptor activity. The composition may be used for modulating blood pressure and blood glucose, inhibiting the accumulation of fat cells, and/or preventing and treating metabolic diseases.

### BACKGROUND

### 1. Technical Field

The present invention relates to demethylzeylasteral and pharmaceutically acceptable salts thereof having anti-obesity effects.

### 2. Background Art

Modern society has shifted to a meat-based diet, resulting in excess calorie intake and insufficient exercise, which has led to a surge in the incidence of metabolic diseases, including obesity, diabetes, hyperlipidemia, non-alcoholic fatty liver, and dyslipidemia.

Obesity is a condition in which excess fat is accumulated due to excess energy intake that exceeds energy expenditure. The pharmacologic mechanisms of current obesity treatments can be broadly categorized into 1) inhibition of fat absorption, 2) promotion of lipolysis and thermogenesis, 3) modulation of appetite and satiety, 4) inhibition of protein metabolism, and 5) modulation of emotions related to food intake. Popular obesity medications include XENICAL™, which inhibits fat absorption, and REDUCTIL™, which stimulates the sympathetic nervous system to suppress appetite. However, XENICAL™ has been reported to cause fatty stools, abdominal pain, vomiting, itching, and liver damage, and REDUCTIL™ has been reported to cause serious cardiovascular side effects, as well as headache, anorexia, insomnia, and constipation.

Diabetes is a disease caused by insufficient secretion or malfunctioning of insulin, and is characterized by hyperglycemia, i.e., high blood glucose, and the excretion of glucose in the urine. Current diabetes treatments include PPAR-γ activators, GLP-1 derivatives, and DPP-IV inhibitors. These conventional treatments, however, have been reported to have side effects such as weight gain and toxicity to the liver, kidneys, muscles, and heart.

Hyperlipidemia is known as a disease in which more fatty substances are present in the blood than necessary, accumulating on the walls of blood vessels and causing inflammation, resulting in cardiovascular diseases such as myocardial infarction, stroke, or cerebral infarction. The conventional treatment for hyperlipidemia is ‘statin’ drugs, which have HMG-COA reductase inhibitory activity, but they are reported to have side effects such as toxicity to the liver and muscles when used for a long period of time.

Currently, there are no excellent treatments for obesity or metabolic diseases in general, and treatments for each specific disease have been reported to have a number of side effects, so there is an urgent need to develop safe treatments for obesity or metabolic diseases with high efficacy and no side effects.

Tripterygium wilfordii is a plant in the family Celastraceae. It is 2 to 3 meters tall, with small branches that are reddish-brown in color, with pointed edges, small oval-shaped trichomes, and greenish-brown, coarse hairs. The flowering season is from May to June and the fruiting season is from August to September. It grows in shrubby woodlands and scrubland on shady, moist, fertile hillsides, valleys, and streams.

Tripterygium regelii is a deciduous yellow vine in the family Celastraceae, also known as Regel's threewingnut, that grows in clusters at the base of mountains, in valleys, or in forests. It grows to about 2 meters, with reddish-brown branches, densely covered with hump-shaped trichomes. The leaves are oblanceolate, 5 to 15 centimeters long, egg-shaped or elliptical, bright green, hairy above the veins on the back, with dull teeth on the edges. It flowers from June to July and bears fruit from September to October. It is known for its insecticidal, anti-inflammatory, and detoxifying properties.

### SUMMARY

The present invention provides a pharmaceutical composition for treating or preventing a metabolic disease.

The present invention provides a food composition for helping to reduce body fat.

The present invention provides a feed composition having a blood pressure and blood sugar regulating effect.
1. A pharmaceutical composition for treating or preventing a metabolic disease comprising a compound represented by Formula 1 or a pharmaceutically acceptable salt thereof:

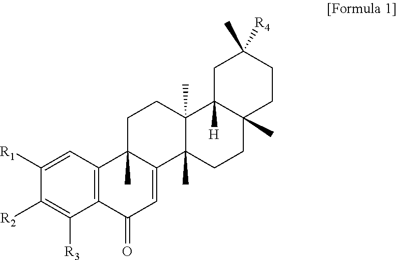

(wherein, R₁ is H, OH or C1 to C5 alkyl, R₂ is H, OH, OCH₃, OCOCH₃ or C1 to C5 alkyl, R₃ is H, CHO, CH₂OH or C1 to C5 alkyl, and R₄ is CH₂OH, COOH or C1 to C5 alkyl.)
2. The pharmaceutical composition of the above 1, wherein the pharmaceutical composition is for administration to a subject selected from the group consisting of a human, a livestock animal, and a pet.
3. The pharmaceutical composition of the above 1, wherein the metabolic disease is selected from the group consisting of obesity, hypertension, hyperlipidemia, and diabetes.
4. The pharmaceutical composition of the above 1, wherein R₁ is H, OH or CH₃, R₂ is H, OH, OCH₃ or OCOCH₃, R₃ is H, CHO, CH₂OH or CH₃, and R₄ is CH₂OH, COOH or CH₃.
5. The pharmaceutical composition of the above 1, wherein the compound represented by Formula 1 is contained in Tripterygium wilfordii, Tripterygium regelii or Tripterygium hypoglaucum.
6. A food composition for helping to reduce body fat comprising a compound represented by Formula 1 or a food-acceptable salt thereof, or an extract of a natural product containing the same.
7. The food composition of the above 6, wherein the food composition helps to modulat blood pressure and blood glucose, and reduce body fat.
8. The food composition of the above 6, wherein R₁ is H, OH, or CH₃, R₂ is H, OH, OCH₃, or OCOCH₃, R₃ is H, CHO, CH₂OH, or CH₃, and R₄ is CH₂OH, COOH, or CH₃.
9. The food composition of the above 6, wherein the natural product is selected from the group consisting of Tripterygium wilfordii, Tripterygium regelii, and Tripterygium hypoglaucum.
10. A feed composition comprising a compound represented by Formula 1, or a feed-acceptable salt thereof, or an extract of a natural product containing the same.
11. The feed composition of the above 10, wherein R₁ is H, OH or CH₃, R₂ is H, OH, OCH₃ or OCOCH₃, R₃ is H, CHO, CH₂OH or CH₃, and R₄ is CH₂OH, COOH or CH₃.
12. The feed composition of the above 10, wherein the natural product is selected from the group consisting of Tripterygium wilfordii, Tripterygium regelii, and Tripterygium hypoglaucum.

The compounds of the present invention, such as those represented by Formula 1, are effective in the treatment and prevention of metabolic diseases such as obesity, diabetes, hyperlipidemia, hypertriglyceridemia, and the like.

The compounds of the present invention, such as those represented by Formula 1, may act as antagonists of glucocorticoid receptors to modulate the transcriptional mechanisms of genes involved in metabolism.

The compounds such as those represented by Formula 1 of the present invention may inhibit the differentiation of adipocytes and promote the breakdown of fat.

### BRIEF DESCRIPTION OF THE DRAWINGS

FIG. 1 is a graph showing the binding affinity of demethylzeylasteral to the glucocorticoid receptor.

FIG. 2 is a graph showing the inhibitory effect of demethylzeylasteral on intracellular glucocorticoid receptor activity.

FIG. 3 is a graph showing the inhibitory effect of demethylzeylasteral on fat accumulation.

FIG. 4 shows the molecular docking between demethylzeylasteral and the glucocorticoid receptor as determined using the AutoDock Vina tool.

FIG. 5 shows the interaction of demethylzeylasteral with the binding site residue of the glucocorticoid receptor as determined using the Pose View tool.

### DETAILED DESCRIPTION

The present invention provides pharmaceutical compositions for treating or preventing metabolic diseases, comprising a compound represented by Formula 1.

In Formula 1, R₁ is H, OH, or C1 to C5 alkyl, R₂ is H, OH, OCH₃, OCOCH₃, or C1 to C5 alkyl, R₃ is H, CHO, CH₂OH, or C1 to C5 alkyl, and R₄ is CH₂OH, COOH, or C1 to C5 alkyl.

In Formula 1, it is preferable that R₁ is H, OH, or CH₃, R₂ is H, OH, OCH₃, or OCOCH₃, R₃ is H, CHO, CH₂OH, or CH₃, and R₄ is CH₂OH, COOH, or CH₃.

More preferably, the compound represented by Formula 1 has the structure of Formula 2 below.

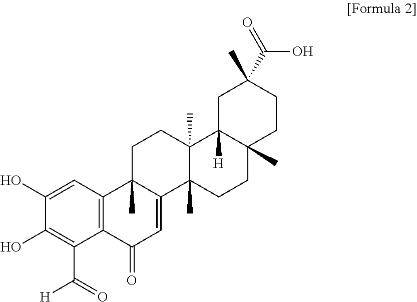

The compound represented by Formula 2 is demethylzeylasteral. It is a type of triterpenoid compound with a molecular formula of C₂₉H₃₆O₆ and a molecular weight of 480.60, and is also named 12-oxodendrobane or (2R,4aS,6aR,6aS,14aS,14bR)-9-formyl-10,11-dihydroxy-2,4a,6a,6a,14a-pentamethyl-8-oxo-1,3,4,5,6,13,14,14b-octahydropicene-2-carboxylic acid.

A pharmaceutical composition comprising a compound represented by Formula 1, or a pharmaceutically acceptable salt thereof may form hydrophobic interactions or hydrogen bonds with a glucocorticoid receptor.

Demethylzeylasteral can be synthesized chemically or isolated from natural products such as Tripterygium wilfordii Hook.f., Tripterygium regelii, and Tripterygium hypoglaucum. When isolating from natural products, solvents such as chloroform, dichloromethane, ethyl acetate, DMSO, or acetone can be used.

The pharmaceutical compositions of the present invention are effective in the treatment and prevention of metabolic diseases. Metabolic diseases refer to conditions that are caused by lifestyle factors such as obesity, lack of exercise, and overnutrition. For example, metabolic diseases include obesity, hypertension, hyperlipidemia, and diabetes. Metabolic diseases can be accompanied by or cause inflammation and cardiovascular diseases (angina, myocardial infarction, stroke, etc.).

A pharmaceutically acceptable salt of a compound of the present invention refers to a salt that is pharmaceutically acceptable as defined in the present invention and has the desired pharmacological activity of the parent compound.

Pharmaceutically acceptable salts may be, for example, acid addition salts or metal salts.

Acid addition salts can be formed from inorganic acids such as hydrochloric, nitric, phosphoric, sulfuric, hydrobromic, hydroiodic, nitrous or phosphorous acid, and non-toxic organic acids such as aliphatic mono- and di-carboxylates, phenyl-substituted alkanoates, hydroxy alkanoates and alkanedioates, aromatic acids, aliphatic and aromatic sulfonic acids. These pharmaceutically non-toxic salts include sulfates, pyrosulfates, bisulfates, sulfites, bisulfites, nitrates, phosphates, mono hydrogen phosphates, dihydrogen phosphates, metaphosphates, pyrophosphates, chlorides, bromides, iodides, fluorides, acetates, propionates, decanoates, caprylates, acrylates, formates, isobutyrates, caprates, heptanoates, propylates, oxalates, malonates, succinates, suberates, sebacates, fumarates, maleates, butyne-1,4-dioate, hexane-1,6-dioate, benzoates, chlorobenzoates, methylbenzoate, dinitrobenzoate, hydroxybenzoates, methoxybenzoates, phthalates, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate or mandelate. For example, acid addition salts can be obtained by dissolving a compound in an excess of aqueous acid solution and precipitating the salt using a hydrated organic solvent, such as methanol, ethanol, acetone or acetonitrile.

The metal salts may be sodium, potassium or calcium salts. Metal salts can be prepared using a base, for example, alkali metal or alkaline earth metal salts can be obtained by dissolving the compound in an excess of alkali metal hydroxide or alkaline earth metal hydroxide solution, filtering the non-dissolved compound salt, and evaporating and/or drying the filtrate.

These salts can be prepared by conventional chemical methods from compounds having a basic or acidic moiety, and a corresponding acid or base.

The pharmaceutical compositions of the present invention may be formulated according to conventional methods in oral formulations such as pills, granules, tablets, capsules, suspensions, emulsions, syrups, aerosols; topicals; suppositories; and sterile injectable solutions.

Examples of carriers, excipients, and diluents that may be included in the composition include, but are not limited to, lactose, dextrose, sucrose, dextrin, maltodextrin, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils. When formulated, it may be prepared using commonly used, but not limited to, excipients or diluents such as fillers, extenders, binders, wetting agents, disintegrants, and surfactants.

Examples of solid formulations for oral administration include, but are not limited to, tablets, pills, powders, granules, capsules and the like, and these solid formulations are prepared by mixing with at least one excipient selected from starch, calcium carbonate, sucrose or lactose, gelatin, and the like. Further, in addition to simple excipients, lubricants such as magnesium stearate and talc may also be used.

Examples of liquid formulations for oral administration include, but are not limited to, suspensions, emulsions, syrups, and the like, and may include a variety of excipients, such as wetting agents, sweeteners, flavors, and preservatives, in addition to the commonly used simple diluents of water or liquid paraffin. Examples of formulations for parenteral administration include sterile aqueous solutions, non-aqueous solutions, suspensions, emulsions, lyophilized preparations, and suppositories. Examples of non-aqueous solvents and suspensions include propylene glycol, polyethylene glycol, vegetable oils including olive oil, and injectable esters such as ethyl oleate. Bases for the suppositories may be witepsol, macrogol, tween 61, cacao butter, Laurin, or glycerogelatin.

The pharmaceutical composition of the present invention is administered in a pharmaceutically effective amount. As used herein, “pharmaceutically effective amount” refers to an amount sufficient to treat a disease at a reasonable benefit/risk ratio applicable to medical treatment, and the effective dose level may be determined based on factors including the type and severity of the patient's condition, the activity of the drug, sensitivity to the drug, time of administration, route of administration and excretion rate, duration of treatment, concomitant medications, and other factors well known in the medical field. The pharmaceutical composition of the present invention may be administered individually or in combination with other conventional therapeutic agents. When administered in combination, the pharmaceutical compositions may be administered sequentially or simultaneously with other conventional therapeutic agents. The pharmaceutical composition may be administered in a single dose or multiple doses. Taking all of the above factors into account, it is important to administer an amount that can achieve the maximum effect with the minimum amount without any adverse side effects, and the amount may be readily determined by a person of ordinary skill in the art.

In the present invention, the effective amount of the pharmaceutical composition may vary depending on the age, gender, and weight of the patient. However, the above amount is not intended to limit the scope of the present invention in any way, as it may be increased or decreased depending on the route of administration, severity of the disease, gender, weight, age and the like.

A subject to which the pharmaceutical composition is administered is not particularly limited, but may preferably be mammals including domestic animal, human, and the like. In the present invention, “domestic animal” refers to any animal useful in human life that has been domesticated and improved by mankind from wildlife.

Examples of domestic animal include, but are not limited to, cattle, horses, mules, donkeys, goats, mountain goats, cotton sheep, deer, pigs, rabbits, and poultry, and examples poultry include, but are not limited to, chickens, turkeys, ducks, ostriches, geese, and quails, as long as they are suitable for raising to obtain livestock products. “Livestock products” refer to meat, milk, eggs, honey and their processed products, raw skin (including raw fur), raw wool, and other livestock products as defined in Article 2 (3) of the Livestock Act, as prescribed by the Ministry of Agriculture and Forestry.

A subject to which the pharmaceutical composition is administered may be pets, including livestock, poultry, and fish.

Further, the present invention provides a food composition comprising a compound represented by the Formula 1 or a food-chemically acceptable salt thereof.

A food composition comprising a compound represented by the above Formula 1 or a food-chemically acceptable salt thereof may form hydrophobic interactions or hydrogen bonds with a glucocorticoid receptor.

A food composition of the present invention includes all forms including functional food, nutritional supplement, health food, and food additives.

A food composition may contain conventional food additives, and their suitability as food additives is determined by the standards and criteria for such items in accordance with the general rules and general test methods of Food Additives Codex approved by Ministry of Food and Drug Safety in Korea, unless otherwise specified.

Items listed in the “Food Additives Codex” include, for example, chemical synthetics such as ketones, glycine, potassium citrate, nicotinic acid and cinnamic acid; natural additives such as persimmon color, licorice extract, crystalline cellulose, kaoliang color and guar gum; mixed formulations such as L-sodium glutamate formulations, alkali agents for noodles, preservative formulations and tar color formulations.

The food composition of the present invention may be provided in the form of a tablet, and the tablet may be formed by mixing the composition with excipients, binders, disintegrants, and other additives, granulating in a conventional manner, and then compressing with a lubricant or the like, or compressing the mixture directly. Furthermore, the food composition in tablet form may contain flavorings or the like as desired.

The food composition of the present invention may be provided in capsule form, for example, such as a hard capsule prepared by mixing the composition with additives such as excipients and filling it into a conventional hard capsule, or a soft capsule prepared by mixing the composition with additives such as excipients and filling it into a capsule base such as gelatin. The soft capsule may contain colorants, preservatives, plasticizers such as glycerin or sorbitol, as desired.

The food composition of the present invention may be provided in pellet form, and may be prepared by mixing the composition with an excipient, a binder, a disintegrant, and/or the like, and molding the mixture using a conventionally known method, and, if necessary, may be coated with white sugar or other coating agents, or the surface may be coated with a material such as starch or talc.

The food composition of the present invention may be provided in granular form, which may be prepared by conventional methods known in the art by mixing the composition with excipients, binders, disintegrating agents, and the like, and flavorings, if desired.

The food composition may be, but is not limited to, a beverage, meat, chocolate, food product, confectionery, pizza, ramen, other noodles, chewing gum, candy, ice cream, alcoholic beverage, vitamin complexes, and dietary supplements.

The present invention may provide a food composition for reducing body fat, controlling blood sugar, controlling blood pressure, and anti-obesity comprising a compound represented by Formula 1 or a food-acceptable salt thereof.

The food composition may further comprise a food-acceptable dietary supplement in addition to the active ingredient.

In another aspect of the present invention, there is provided a feed composition comprising a compound represented by Formula 1, or a feed-acceptable salt thereof.

The feed composition comprising a compound represented by Formula 1, or a feed-acceptable salt thereof, may form hydrophobic interactions or hydrogen bonds with a receptor of a glucocorticoid.

As used in the present invention, the term “feed” refers to any natural or artificial diet, meal, or the like, or any ingredient of said meal, which is intended for or adapted to be eaten, consumed, and digested by an animal.

More specifically, the present invention may provide a feed composition for reducing body fat, controlling blood sugar, controlling blood pressure, and anti-obesity comprising a compound represented by Formula 1 or a feed-acceptable salt thereof.

In the present invention, the feed may be a feed for reptiles, fish, birds, or mammals. Preferably, it may be a feed for livestock or aquatic organisms that are suitable for breeding due to their wild nature being acclimated, and can contribute to increasing the income of farmers, as defined in Article 2, Paragraph 1 of the Livestock Industry Act and Article 2, Subparagraphs of the Enforcement Decree of the same Act.

Formula 1 of the present invention may have substituents as shown in Table 1 below:

TABLE 1

| No. | R₁ | R₂ | R₃ | R₄ |
|---|---|---|---|---|
| 1 | H | H | H | CH₃ |
| 2 | H | H | H | CH₂OH |
| 3 | H | H | H | COOH |
| 4 | H | H | CH₃ | CH₃ |
| 5 | H | H | CH₃ | CH₂OH |
| 6 | H | H | CH₃ | COOH |
| 7 | H | H | CHO | CH₃ |
| 8 | H | H | CHO | CH₂OH |
| 9 | H | H | CHO | COOH |
| 10 | H | H | CH₂OH | CH₃ |
| 11 | H | H | CH₂OH | CH₂OH |
| 12 | H | H | CH₂OH | COOH |
| 13 | H | OH | H | CH₃ |
| 14 | H | OH | H | CH₂OH |
| 15 | H | OH | H | COOH |
| 16 | H | OH | CH₃ | CH₃ |
| 17 | H | OH | CH₃ | CH₂OH |
| 18 | H | OH | CH₃ | COOH |
| 19 | H | OH | CHO | CH₃ |
| 20 | H | OH | CHO | CH₂OH |
| 21 | H | OH | CHO | COOH |
| 22 | H | OH | CH₂OH | CH₃ |
| 23 | H | OH | CH₂OH | CH₂OH |
| 24 | H | OH | CH₂OH | COOH |
| 25 | H | OCH₃ | H | CH₃ |
| 26 | H | OCH₃ | H | CH₂OH |
| 27 | H | OCH₃ | H | COOH |
| 28 | H | OCH₃ | CH₃ | CH₃ |
| 29 | H | OCH₃ | CH₃ | CH₂OH |
| 30 | H | OCH₃ | CH₃ | COOH |
| 31 | H | OCH₃ | CHO | CH₃ |
| 32 | H | OCH₃ | CHO | CH₂OH |
| 33 | H | OCH₃ | CHO | COOH |
| 34 | H | OCH₃ | CH₂OH | CH₃ |
| 35 | H | OCH₃ | CH₂OH | CH₂OH |
| 36 | H | OCH₃ | CH₂OH | COOH |
| 37 | H | OCOCH₃ | H | CH₃ |
| 38 | H | OCOCH₃ | H | CH₂OH |
| 39 | H | OCOCH₃ | H | COOH |
| 40 | H | OCOCH₃ | CH₃ | CH₃ |
| 41 | H | OCOCH₃ | CH₃ | CH₂OH |
| 42 | H | OCOCH₃ | CH₃ | COOH |
| 43 | H | OCOCH₃ | CHO | CH₃ |
| 44 | H | OCOCH₃ | CHO | CH₂OH |
| 45 | H | OCOCH₃ | CHO | COOH |
| 46 | H | OCOCH₃ | CH₂OH | CH₃ |
| 47 | H | OCOCH₃ | CH₂OH | CH₂OH |
| 48 | H | OCOCH₃ | CH₂OH | COOH |
| 49 | CH₃ | H | H | CH₃ |
| 50 | CH₃ | H | H | CH₂OH |
| 51 | CH₃ | H | H | COOH |
| 52 | CH₃ | H | CH₃ | CH₃ |
| 53 | CH₃ | H | CH₃ | CH₂OH |
| 54 | CH₃ | H | CH₃ | COOH |
| 55 | CH₃ | H | CHO | CH₃ |
| 56 | CH₃ | H | CHO | CH₂OH |
| 57 | CH₃ | H | CHO | COOH |
| 58 | CH₃ | H | CH₂OH | CH₃ |
| 59 | CH₃ | H | CH₂OH | CH₂OH |
| 60 | CH₃ | H | CH₂OH | COOH |
| 61 | CH₃ | OH | H | CH₃ |
| 62 | CH₃ | OH | H | CH₂OH |
| 63 | CH₃ | OH | H | COOH |
| 64 | CH₃ | OH | CH₃ | CH₃ |
| 65 | CH₃ | OH | CH₃ | CH₂OH |
| 66 | CH₃ | OH | CH₃ | COOH |
| 67 | CH₃ | OH | CHO | CH₃ |
| 68 | CH₃ | OH | CHO | CH₂OH |
| 69 | CH₃ | OH | CHO | COOH |
| 70 | CH₃ | OH | CH₂OH | CH₃ |
| 71 | CH₃ | OH | CH₂OH | CH₂OH |
| 72 | CH₃ | OH | CH₂OH | COOH |
| 73 | CH₃ | OCH₃ | H | CH₃ |
| 74 | CH₃ | OCH₃ | H | CH₂OH |
| 75 | CH₃ | OCH₃ | H | COOH |
| 76 | CH₃ | OCH₃ | CH₃ | CH₃ |
| 77 | CH₃ | OCH₃ | CH₃ | CH₂OH |
| 78 | CH₃ | OCH₃ | CH₃ | COOH |
| 79 | CH₃ | OCH₃ | CHO | CH₃ |
| 80 | CH₃ | OCH₃ | CHO | CH₂OH |
| 81 | CH₃ | OCH₃ | CHO | COOH |
| 82 | CH₃ | OCH₃ | CH₂OH | CH₃ |
| 83 | CH₃ | OCH₃ | CH₂OH | CH₂OH |
| 84 | CH₃ | OCH₃ | CH₂OH | COOH |
| 85 | CH₃ | OCOCH₃ | H | CH₃ |
| 86 | CH₃ | OCOCH₃ | H | CH₂OH |
| 87 | CH₃ | OCOCH₃ | H | COOH |
| 88 | CH₃ | OCOCH₃ | CH₃ | CH₃ |
| 89 | CH₃ | OCOCH₃ | CH₃ | CH₂OH |
| 90 | CH₃ | OCOCH₃ | CH₃ | COOH |
| 91 | CH₃ | OCOCH₃ | CHO | CH₃ |
| 92 | CH | OCOCH₃ | CHO | CH₂OH |
| 93 | CH₃ | OCOCH₃ | CHO | COOH |
| 94 | CH₃ | OCOCH₃ | CH₂OH | CH₃ |
| 95 | CH₃ | OCOCH₃ | CH₂OH | CH₂OH |
| 96 | CH₃ | OCOCH₃ | CH₂OH | COOH |
| 97 | OH | H | H | CH₃ |
| 98 | OH | H | H | CH₂OH |
| 99 | OH | H | H | COOH |
| 100 | OH | H | CH₃ | CH₃ |
| 101 | OH | H | CH₃ | CH₂OH |
| 102 | OH | H | CH₃ | COOH |
| 103 | OH | H | CHO | CH₃ |
| 104 | OH | H | CHO | CH₂OH |
| 105 | OH | H | CHO | COOH |
| 106 | OH | H | CH₂OH | CH₃ |
| 107 | OH | H | CH₂OH | CH₂OH |
| 108 | OH | H | CH₂OH | COOH |
| 109 | OH | OH | H | CH₃ |
| 110 | OH | OH | H | CH₂OH |
| 111 | OH | OH | H | COOH |
| 112 | OH | OH | CH₃ | CH₃ |
| 113 | OH | OH | CH₃ | CH₂OH |
| 114 | OH | OH | CH₃ | COOH |
| 115 | OH | OH | CHO | CH₃ |
| 116 | OH | OH | CHO | CH₂OH |
| 117 | OH | OH | CHO | COOH |
| 118 | OH | OH | CH₂OH | CH₃ |
| 119 | OH | OH | CH₂OH | CH₂OH |
| 120 | OH | OH | CH₂OH | COOH |
| 121 | OH | OCH₃ | H | CH₃ |
| 122 | OH | OCH₃ | H | CH₂OH |
| 123 | OH | OCH₃ | H | COOH |
| 124 | OH | OCH₃ | CH₃ | CH₃ |
| 125 | OH | OCH₃ | CH₃ | CH₂OH |
| 126 | OH | OCH₃ | CH₃ | COOH |
| 127 | OH | OCH₃ | CHO | CH₃ |
| 128 | OH | OCH₃ | CHO | CH₂OH |
| 129 | OH | OCH₃ | CHO | COOH |
| 130 | OH | OCH₃ | CH₂OH | CH₃ |
| 131 | OH | OCH₃ | CH₂OH | CH₂OH |
| 132 | OH | OCH₃ | CH₂OH | COOH |
| 133 | OH | OCOCH₃ | H | CH₃ |
| 134 | OH | OCOCH₃ | H | CH₂OH |
| 135 | OH | OCOCH₃ | H | COOH |
| 136 | OH | OCOCH₃ | CH₃ | CH₃ |
| 137 | OH | OCOCH₃ | CH₃ | CH₂OH |
| 138 | OH | OCOCH₃ | CH₃ | COOH |
| 139 | OH | OCOCH₃ | CHO | CH₃ |
| 140 | OH | OCOCH₃ | CHO | CH₂OH |
| 141 | OH | OCOCH₃ | CHO | COOH |
| 142 | OH | OCOCH₃ | CH₂OH | CH₃ |
| 143 | OH | OCOCH₃ | CH₂OH | CH₂OH |
| 144 | OH | OCOCH₃ | CH₂OH | COOH |

Hereinafter, the present invention will be described in detail with reference to examples.

### EXAMPLE

### Example 1

Demethylzeylasteral used in the Examples was 10 mg of Demethylzeylasteral (CAS No. 107316-88-1, product number 28595, Cayman Chemical, USA) dissolved in DMSO.

### Example 1: Measurement of Binding Affinity of Demethylzeylasteral to Glucocorticoid Receptor

In Example 1, a fluorescence polarization competition assay was performed using POLARSCREEN™ Glucocorticoid Receptor Competitor Assay Kit, Red (ThermoFisher Scientific, USA) to measure the binding affinity of demethylzeylasteral to glucocorticoid receptor.

Fluorescence polarization competition assay was performed by mixing a fluormone tracer, a fluorescent substance that binds to purified glucocorticoid receptors, with demethylzeylasteral. The principle of the assay is to determine the binding affinity of a small molecule compound to glucocorticoid receptor by measuring the decrease in polarization caused by the failure of the fluormone tracer to bind to the glucocorticoid receptor in the presence of a small molecule compound that competitively binds to the glucocorticoid receptor.

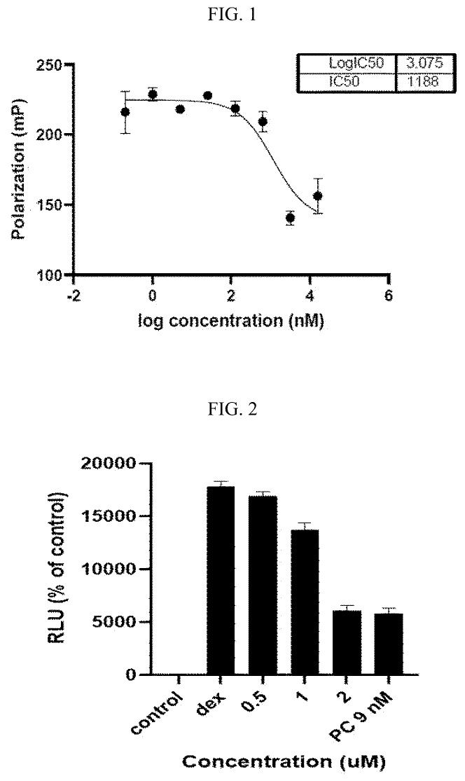

More specifically, 2,585 nM glucocorticoid receptor, 1.4 nM fluormone tracer, and demethylzeylasteral (maximum concentration of 16 μM, 5-fold serial dilution) were prepared, mixed in a 384-well black plate, reacted for 3 hours at room temperature, and fluorescence polarization (mP) was measured at room temperature using Hidex Sense (Hidex, Finland). The excitation wavelength used for the measurement was 535 nm, and the emission wavelength was 590 nm. This experiment was performed three times using the same method and the average value was calculated. A dose-response curve was derived using the measured values, and the IC50 value was calculated by finding the concentration of the sample corresponding to half of the highest measured value. The result is shown in FIG. 1.

According to this Example, the IC50 was determined to be 1188 nM based on the calculated binding affinity of demethylzeylasteral for the glucocorticoid receptor.

### Example 2: Inhibitory Effect of Demethylzeylasteral on Intracellular Glucocorticoid Receptor Activity

A luciferase assay was performed to measure the inhibitory effect of demethylzeylasteral on intracellular glucocorticoid receptor activity. In this Example, transfected cells GR-GAL4 Reporter (Luc)-HEK293 (BPS Bioscience, USA) containing a recombinant gene in which luciferase is recombined into the promoter site of GRE were used. Transformed cells were seeded at 30,000 cells per well in 96 wells and stabilized for 16 hours. The cells were then treated with demethylzeylasteral at different concentrations, or mifepristone at a concentration of 9 nM as a positive control. After 1 hour, the cells were treated with dexamethasone, a glucocorticoid agonist, at a concentration of 20 nM and incubated for 24 hours. 100 μL of ONE-STEP™ Luciferase Assay System (BPS Bioscience, USA) was added per well and reacted for 10 minutes, and then luminescence was measured using SpectraMax iD3 (Molecular Devices, USA). The result is shown in FIG. 2.

It was confirmed that demethylzeylasteral inhibited intracellular glucocorticoid receptor activity in a concentration-dependent manner.

### Example 3: Inhibitory Effect of Demethylzeylasteral on Fat Accumulation

In Example 3, preadipocyte were differentiated and Oil Red O staining was performed to analyze the inhibitory effect of demethylzeylasteral on fat accumulation.

To evaluate the effect of demethylzeylasteral on the differentiation and growth of adipocytes induced by methylisobutylxanthine (MDI), 3T3-L1 cells, which are preadipocytes, were seeded in 48-well plates and cultured in DMEM (Dulbecco-modified Eagle medium) medium supplemented with 10% bovine calf serum (BCS) and 10 ml/L antibiotic-antimycotic at 37° C. in a 10% CO² incubator until confluent.

To differentiate 3T3-L1 cells grown to a confluent state into adipocytes, they were cultured in DMEM medium supplemented with MDI {0.5 mM isobutyl-methylxanthine, 1 μM dexamethasone, and 5 μg/mL insulin} for 48 h, then cultured in DMEM medium supplemented with 5 μg/mL insulin for 48 h, and then cultured in a medium containing 10% fetal bovine serum for 48 h.

From the first day of differentiation of 3T3-L1 cells by adding MDI, the cultures were treated with demethylzeylasteral at concentrations of 0.5 or 1 μM every 48 hours when the medium was replaced. Demethylzeylasteral was dissolved in DMSO before use. The cells were incubated for a total of 6 days, and the cultures were removed at which point differentiation was complete, followed by Oil Red O staining. The cells were fixed with 35 to 40% formaldehyde for 15 minutes, washed with PBS, and stained with Oil Red O solution for 20 minutes. After completion of staining, the cells were washed four times with PBS, dissolved in isopropanol (2-propanol), and O.D. values were measured at 515 nm. The result is shown in FIG. 3.

Treating 3T3-L1 preadipocytes with demethylzeylasteral resulted in a reduction of intracellular fat accumulation in a concentration-dependent manner.

### Example 4: In Silico Molecular Docking Experiment Between Demethylzeylasteral and Glucocorticoid Receptor

Molecular docking is a method for predicting interactions between molecules (proteins, compounds, etc.). This can reproduce in silico the steric structure motions and interactions between molecules in aqueous solution and calculate the binding energies between molecules allowing us to predict the intermolecular binding sites.

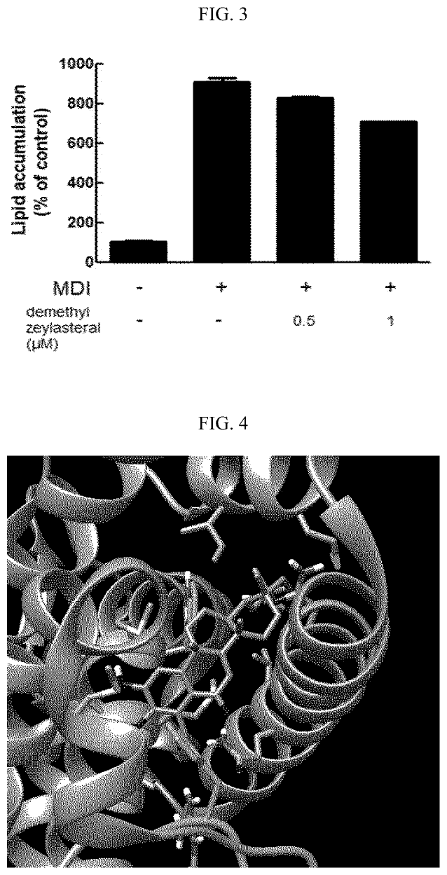

The three-dimensional structure of the glucocorticoid receptor was obtained from the Protein Data Bank (PDB) database, and the three-dimensional structure of demethylzeylasteral was obtained from the PubChem database. Molecular docking was performed using the AutoDock Vina tool in the UCSF Chimera v1.15 program. The results verified that demethylzeylasteral stably binds within the binding site of the glucocorticoid receptor (FIG. 4).

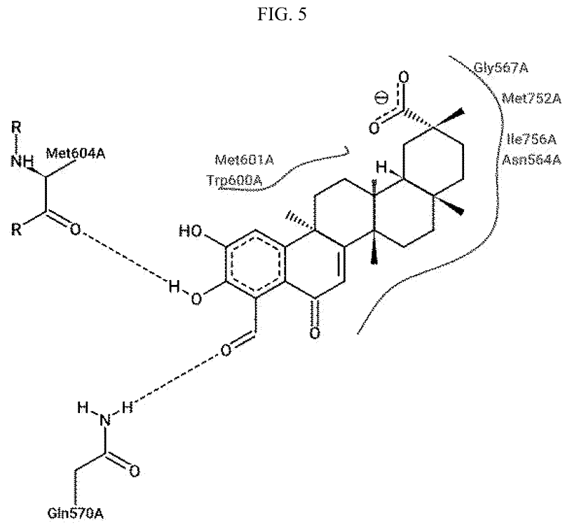

To confirm the intermolecular interactions formed in the binding site, the PoseView tool of the ProteinPlus program was used to observe the interaction between the binding site residue of the glucocorticoid receptor and demethylzeylasteral (FIG. 5). As a result, it was found that the OH and CHO of C1 ring of demethylzeylasteral form hydrogen bonds with the Met604 and Gln570 residues of the protein, respectively. Furthermore, it was found that residues in the C3 to C5 rings form hydrophobic interactions with residues in the binding site. Accordingly, it was determined that the hydrogen bond between OH and CHO of C1 ring is the main reason for the strong binding of demethylzeylasteral to the glucocorticoid receptor.

1. A method for treating a metabolic disease, the method comprising administering a composition comprising a compound represented by Formula 1 or a pharmaceutically acceptable salt thereof to a subject in need thereof: 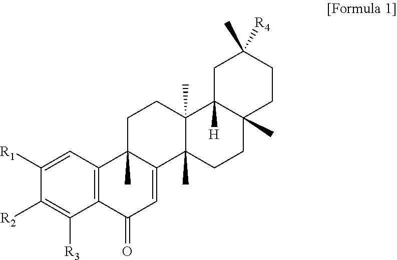
wherein R₁ is H, OH or C1 to C5 alkyl, R₂ is H, OH, OCH₃, OCOCH₃ or C1 to C5 alkyl, R₃ is H, CHO, CH₂OH or C1 to C5 alkyl, and R₄ is CH₂OH, COOH or C1 to C5 alkyl.

2. The method of claim 1, wherein the subject is selected from the group consisting of a human, a livestock animal, and a pet.

3. The method of claim 1, wherein the metabolic disease is selected from the group consisting of obesity, hypertension, hyperlipidemia, and diabetes.

4. The method of claim 1, wherein R₁ is H, OH or CH₃, R₂ is H, OH, OCH₃ or OCOCH₃, R₃ is H, CHO, CH₂OH or CH₃, and R₄ is CH₂OH, COOH or CH₃.

5. The method of claim 1, wherein the composition comprises Tripterygium wilfordii extract, Tripterygium regelii extract or Tripterygium hypoglaucum extract containing the compound represented by Formula 1.

6. A method for reducing body fat, the method comprising administering a composition comprising a compound represented by Formula 1 or a salt thereof to a subject in need thereof: 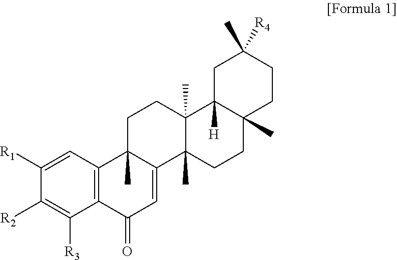
wherein R₁ is H, OH or C1 to C5 alkyl, R₂ is H, OH, OCH₃, OCOCH₃ or C1 to C5 alkyl, R₃ is H, CHO, CH₂OH or C1 to C5 alkyl, and R₄ is CH₂OH, COOH or C1 to C5 alkyl.

7. (canceled)

8. The method of claim 6, wherein R₁ is H, OH, or CH₃, R₂ is H, OH, OCH₃, or OCOCH₃, R₃ is H, CHO, CH₂OH, or CH₃, and R₄ is CH₂OH, COOH, or CH₃.

9. The method of claim 6, wherein the composition comprises a natural extract containing the compound represented by Formula 1 or the salt thereof, and the natural extract is selected from the group consisting of Tripterygium wilfordii extract, Tripterygium regelii extract, and Tripterygium hypoglaucum extract.

10-12. (canceled)

13. The method of claim 6, wherein the composition is a food composition, and the salt is a food-acceptable salt thereof.

14. The method of claim 6, wherein the composition is a pharmaceutical composition, and the salt is a pharmaceutically acceptable salt thereof.

15. A method for modulating blood pressure and blood glucose, the method comprising administering a composition comprising a compound represented by Formula 1 or a salt thereof to a subject in need thereof: 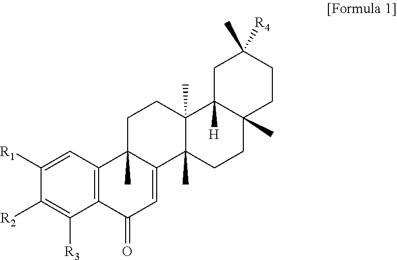
wherein R₁ is H, OH or C1 to C5 alkyl, R₂ is H, OH, OCH₃, OCOCH₃ or C1 to C5 alkyl, R₃ is H, CHO, CH₂OH or C1 to C5 alkyl, and R₄ is CH₂OH, COOH or C1 to C5 alkyl.

16. The method of claim 15, wherein the subject is selected from the group consisting of a human, a livestock animal, and a pet.

17. The method of claim 15, wherein R₁ is H, OH or CH₃, R₂ is H, OH, OCH₃ or OCOCH₃, R3 is H, CHO, CH₂OH or CH₃, and R₄ is CH₂OH, COOH or CH₃.

18. The method of claim 15, wherein the composition comprising Tripterygium wilfordii extract, Tripterygium regelii extract or Tripterygium hypoglaucum extract containing the compound represented by Formula 1.
